(19) United States
(12) Patent Application Publication
(10) Pub. No.: US 2024/0288372 A1
(43) Pub. Date: Aug. 29, 2024

(54) GENE ANALYSIS METHOD AND KIT FOR GENE ANALYSIS

(71) Applicant: HITACHI, LTD., Tokyo (JP)

(72) Inventors: Takahiro Ando, Tokyo (JP); Takahide Yokoi, Tokyo (JP); Chihiro Manri, Tokyo (JP); Takeshi Ishida, Tokyo (JP)

(73) Assignee: HITACHI, LTD., Tokyo (JP)

(21) Appl. No.: 18/426,589

(22) Filed: Jan. 30, 2024

(30) Foreign Application Priority Data
Feb. 28, 2023 (JP) 2023-029502

(51) Int. Cl. G01N 21/64; C12Q 1/6806; G01N 27/447

(52) U.S. Cl. CPC G01N 21/6486; C12Q 1/6806; G01N 27/447

(57) ABSTRACT

Provided is a gene analysis method including performing a single-base extension reaction using a primer for a single-base extension reaction for detection of a target nucleotide sequence, and a substrate for a single-base extension reaction having a fluorescent dye; subjecting a reaction product of the single-base extension reaction to electrophoresis; and measuring mobility of the electrophoresis and fluorescence intensity of the fluorescent dye, and detecting a wild type and a mutant of the target nucleotide sequence based on the fluorescence intensity. Also provided is a kit for use in the gene analysis method disclosed herein.

### CROSS-REFERENCE TO RELATED APPLICATION

This application claims priority to JP Patent Application No. 2023-029502 filed on Feb. 28, 2023, the content of which is incorporated herein by reference in its entirety.

### SEQUENCE LISTING

The instant application contains a Sequence Listing which has been submitted electronically in XML format and is hereby incorporated by reference in its entirety. Said XML copy, created on Jan. 22, 2024, is named 0723881650SL.xml and is 2,120 bytes in size.

### BACKGROUND OF THE INVENTION

### 1. Field of the Invention

The present invention relates to a gene analysis method using fragment analysis of electrophoresis, and a kit for gene analysis for use in the method.

### 2. Description of the Related Art

Cancer research has progressed rapidly in recent years, and importance of detecting genetic mutations derived from tumors by a genetic analysis technique has increased. In particular, a test for performing medical diagnosis by detecting genetic mutations derived from tumors in blood is called liquid biopsy, and is expected to be applied to early diagnosis of cancer, optimization of treatment selection after surgery, monitoring of residual tumor, and the like. In a search for tumor-associated genetic mutations that serve as biomarkers, it is currently possible to use a next-generation sequencer (NGS) to perform large-scale and high-speed analysis, and it is becoming easier to extract items of genetic mutations required for liquid biopsy. Therefore, for example, in cancer diagnosis, there is a trend to increase versatility as a testing technique by measuring genetic mutations derived from tumors identified based on results of comprehensive analysis in NGS by a technique of detecting genetic mutations more advantageous than NGS in terms of cost and detection sensitivity.

An example of a lower-cost technique of detecting genetic mutations than NGS is fragment analysis using electrophoresis, for example, capillary electrophoresis (CE). As shown in FIG. 1, for each target gene sequence 110, a selective primer 111 having a different molecular weight (base length) is designed so as to change mobility in electrophoresis, and, by a polymerase reaction, a ddNTP 112 modified with four fluorescent dyes is applied to the 3′ end of the selective primer 111 corresponding to a genetic mutation by a single-base extension reaction. The double-stranded DNA is converted into single-strands by formamide treatment and thermal denaturation, and the fluorescent dye at the 3′ end is fluorescently detected to identify the genetic mutation. For example, when the genetic mutation EGFR L858R, which is found in many lung cancer patients, is targeted, the 858th base of the EGFR gene is mutated from leucine (L: CUG) to arginine (R: CGG), and when a mutant single-base extension reaction product is generated, as compared to when only a wild-type single-base extension reaction product is present, a fluorescence signal derived from the mutant is observed at a specific electrophoresis detection timing (electrophoretic mobility).

Dias-Santagata, D. et al. (EMBO Molecular Medicine, Vol. 2, pp. 146-158 (2010)) using this method also shows detection of the genetic mutation EGFR L858R. It shows the fact that by utilizing this method, a target tumor-derived gene sequence can be selectively enriched by a multiplex polymerase chain reaction (PCR), and then 120 known genetic mutations in 13 oncogenes can be detected. However, since the upper limit of a migration length at which a selective primer can be separated by electrophoresis is restricted to about 120 bases, the number of genetic mutations that are simultaneously detected per run is several, and intensity of a fluorescence signal varies depending on the fluorescent dye for specifying the genetic mutation, so that quantitativity is not assured. In addition, also in Sanchez, J. J. et al., Electrophoresis, Vol. 27, pp. 1713-1724 (2006), when 52 types of single nucleotide polymorphisms are detected, it is contrived that 29 types and 23 types are separately detected per run.

With respect to the number of genetic mutations that can be simultaneously detected, the present inventors have recently linked an interstrand-crosslinked double-stranded DNA to a selective primer, and stably extend an electrophoretic distance to 120 bp or more, thereby making it possible to increase the number of genetic mutations that can be simultaneously detected by using an electrophoretic region that has not been effectively utilized. Furthermore, as a method of quantifying a small amount of genetic mutations, WO 2016/172571 A proposes an approach of quantitative measurement in which, for example, when detecting genetic mutations in the above-described single-base extension reaction, by changing the concentration of a chain termination reagent for an extended primer (selective primer), the detection limit of a small amount of nucleic acid species present at a low frequency or copy number in a mixture is improved. As described above, with respect to a target gene derived from cancer, an analysis method capable of quantitatively measuring a small amount of mutations by multiplex simultaneous detection has been developed so far.

### SUMMARY OF THE INVENTION

As a method of a cancer diagnosis technique, for example, a technique of detecting known genetic mutations by a method using electrophoresis such as fragment analysis using capillary electrophoresis is considered. As a base length width capable of fragment analysis is increased by improvement of an apparatus for performing capillary electrophoresis, a length of a reaction product can also be increased, so that the number of targets of genetic mutation that can be detected is also increased. As the number of target genes increases, coverage of a gene area increases, which leads to an increase in accuracy of cancer diagnosis. At that time, for example, when an analyzable base length width is increased to 500 bp and the number of targets of genes to be detected is set to 100, a base length width that can be used per target to distinguish by electrophoretic mobility is 5 bp. However, in the case where gene targets that are adjacent as mobility at the time of electrophoresis are detected by labeling with a fluorescent dye, there is a problem that overlapping of the fluorescence signals leads to impairment of quantitativity of mutant genes. Overlapping of fluorescence intensity occurs, which could not be observed in the case of measuring at most 20 to 30 target genes in one run as in the previous research examples, and thus it is necessary to contrive positioning of the targets (primer design).

Therefore, as a result of intensive studies, the present inventors have found that the amount of mutant genes can be quantitatively measured by setting labeling fluorescent dyes to a wild type and a mutant of gene targets that are adjacent as mobility of electrophoresis to be different and eliminating overlapping of fluorescence signals. Furthermore, the present inventors have found that by setting (designing) a primer length (mobility) according to a content ratio of a mutant to be quantified, it is possible to detect more items with adjacent primers while bringing the items as close as possible.

Accordingly, the present invention encompasses the following aspects:

[1] In one aspect, the present invention provides a gene analysis method including: performing a single-base extension reaction using a primer for a single-base extension reaction for detection of a target nucleotide sequence, and a substrate for a single-base extension reaction having a fluorescent dye; subjecting a reaction product of the single-base extension reaction to electrophoresis; and measuring mobility of the electrophoresis and fluorescence intensity of the fluorescent dye, and detecting a wild type and a mutant of the target nucleotide sequence based on the fluorescence intensity, wherein the primer for a single-base extension reaction includes two or more primers having different sequences and different lengths in order to detect two or more target nucleotide sequences, the substrate for a single-base extension reaction having a fluorescent dye includes two or more substrates having different fluorescent dyes, and for the target nucleotide sequences from which the reaction products of the single-base extension reaction that are adjacent as mobility of electrophoresis when measuring the fluorescence intensity are derived, a combination of the primer for a single-base extension reaction and the substrate for a single-base extension reaction is designed so that a fluorescent dye of a substrate incorporated by a single-base extension reaction of a wild type of a first target nucleotide sequence and a fluorescent dye of a substrate incorporated by a single-base extension reaction of a mutant of a second target nucleotide sequence are different.

[2] In another aspect, the present invention provides a gene analysis method including: performing a single-base extension reaction using a primer for a single-base extension reaction for detection of a target nucleotide sequence, and a substrate for a single-base extension reaction having a fluorescent dye; subjecting a reaction product of the single-base extension reaction to electrophoresis; and measuring mobility of the electrophoresis and fluorescence intensity of the fluorescent dye, and detecting a wild type and a mutant of the target nucleotide sequence based on the fluorescence intensity, wherein the primer for a single-base extension reaction includes two or more primers having different sequences and different lengths in order to detect two or more target nucleotide sequences, the substrate for a single-base extension reaction having a fluorescent dye includes two or more substrates having different fluorescent dyes, and for the target nucleotide sequences from which the reaction products of the single-base extension reaction that are adjacent as mobility of electrophoresis when measuring the fluorescence intensity are derived, the primer for a single-base extension reaction is designed so as to adjust a difference in the mobility of the electrophoresis according to a content ratio of the mutant to the wild type of the target nucleotide sequence.

[3] In yet another aspect, the present invention provides a kit for gene analysis for use in the method according to [1], including: a primer for a single-base extension reaction for detection of a target nucleotide sequence; and a substrate for a single-base extension reaction having a fluorescent dye, wherein the primer for a single-base extension reaction includes two or more primers having different sequences and different lengths in order to detect two or more target nucleotide sequences, the substrate for a single-base extension reaction having a fluorescent dye includes two or more substrates having different fluorescent dyes, and for the target nucleotide sequences from which reaction products of a single-base extension reaction that are adjacent as mobility of electrophoresis are derived, a combination of the primer for a single-base extension reaction and the substrate for a single-base extension reaction is designed so that a fluorescent dye of a substrate incorporated by a single-base extension reaction of a wild type of the first target nucleotide sequence and a fluorescent dye of a substrate incorporated by a single-base extension reaction of a mutant of the second target nucleotide sequence are different.

[4] In further another aspect, the present invention provides a kit for gene analysis for use in the method according to [2], including: a primer for a single-base extension reaction for detection of a target nucleotide sequence; and a substrate for a single-base extension reaction having a fluorescent dye, wherein the primer for a single-base extension reaction includes two or more primers having different sequences and different lengths in order to detect two or more target nucleotide sequences, the substrate for a single-base extension reaction having a fluorescent dye includes two or more substrates having different fluorescent dyes, and for the target nucleotide sequences from which reaction products of a single-base extension reaction that are adjacent as mobility of electrophoresis are derived, the primer for a single-base extension reaction is designed so as to adjust a difference in the mobility of the electrophoresis according to the content ratio of the mutant to the wild type of the target nucleotide sequence.

According to the present invention, it is possible to simultaneously detect more gene targets. A content ratio of a target gene sequence can be quantitatively determined from magnitude of fluorescence intensity, and an abundance ratio of a mutation to a wild type required for cancer diagnosis or a frequency of genetic mutation can be quantified. Problems, configurations, and effects other than those described above will be clarified in the following description of embodiments.

### BRIEF DESCRIPTION OF THE DRAWINGS

FIG. 1 is an explanatory view of fragment analysis using capillary electrophoresis in quantitative measurement of a ratio of mutants to wild types of a plurality of target nucleotide sequences using a single-base extension reaction.

FIG. 2 is an explanatory view showing a state in which fluorescence signals of fluorescent dyes labeled to wild types and mutants of target #1 and target #2 overlap each other because base lengths (i.e. electrophoretic mobility) of primers for detection of target #1 and target #2 are close.

FIG. 3 is an explanatory view showing a state in which there is no overlapping of fluorescence signals because base lengths (i.e. electrophoretic mobility) of primers for detection of target #1 and target #2 are separated from each other, and even when base lengths of primers for detection of target #1 and target #3 are close to each other, types of fluorescent dyes labeled to wild types and mutants of target #1 and target #3 are different from each other.

FIG. 4 is a diagram explaining a correspondence between distance in terms of electrophoretic mobility (base length) and percentage as a fluorescence intensity peak ratio when a fluorescence signal of a labeling fluorescent dye detected by capillary electrophoresis is assumed to be a Gaussian function.

FIG. 5 is a diagram showing results of fluorescence signals of a wild type and a mutant when the genetic mutation EGFR L858R is targeted; The lower part (A) is a diagram showing superposition of a shape of the same fluorescence signal when it is assumed that another adjacent gene target primer is separated by 2 bp in base length; The lower part (B) is a diagram showing superposition of a shape of the same fluorescence signal when it is assumed that another adjacent gene target primer is separated by 5 bp in base length.

### DESCRIPTION OF THE PREFERRED EMBODIMENTS

Hereinafter, an example of embodiments of the present invention will be described with reference to the drawings.

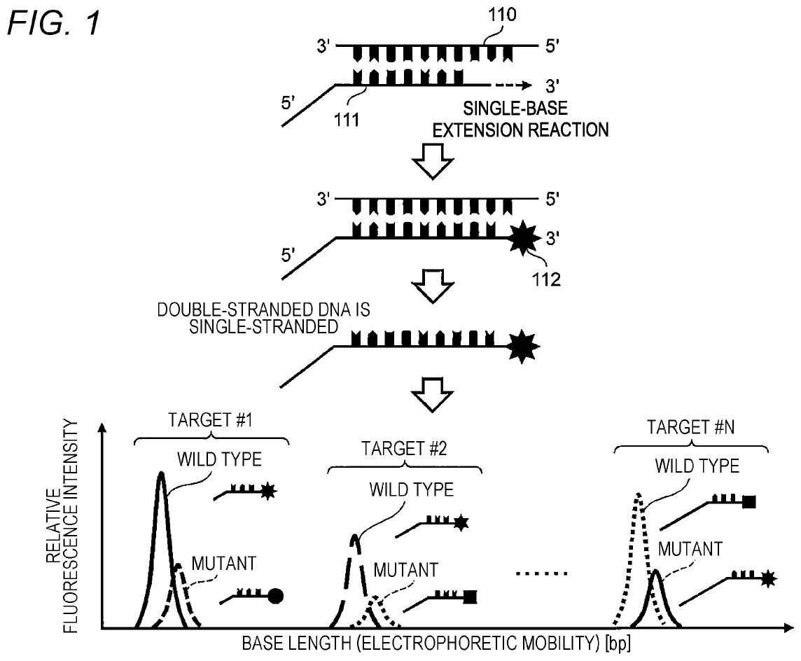

FIG. 1 is an explanatory view of fragment analysis using capillary electrophoresis in quantitative measurement of a ratio of mutants to wild types of a plurality of target genes using a single-base extension reaction. Using a target gene sequence 110 as a template, a selective primer 111 having a different base length (molecular weight) for each sequence (mutation) may be designed so that mobility of electrophoresis of a single-base extension reaction product obtained using the selective primer 111 changes. By a polymerase reaction, a ddNTP 112 which is modified with fluorescent dyes of four colors (four types) corresponding to adenine (A), cytosine (C), guanine (G), and thymine (T) may be added to the 3′ end of the selective primer 111 as a terminal base corresponding to a genetic mutation by a single-base extension reaction (labels are four types for A, G, C, and T). The double-stranded DNA is converted into single-strands by formamide treatment and thermal denaturation, and the fluorescent dye at the 3′ end may be fluorescently detected to identify the genetic mutation. The above process is a general fragment analysis. Previous research examples have shown that 20 or more known genetic mutations can be detected by selective primers having different base lengths. At this time, by designing a primer, not only a gene sequence having a specific length can be detected, but also a single nucleotide polymorphism in which only a single base is mutated can be detected. Alternatively, since insertion and deletion, which are a type of genetic mutation, can also be detected on the same principle, the scope of application of the present invention is general fragment analysis using a ddNTP modified with a fluorescent dye. In the conventional art, as shown in the lower part of FIG. 1, primers for detection of adjacent target nucleotide sequences are designed so as to be separated by at least 4 bases in length as mobility so that each target nucleotide sequence can be distinguished as mobility of electrophoresis. When arranged in this way, since the range of electrophoresis is restricted, the number of items to be detected was also restricted.

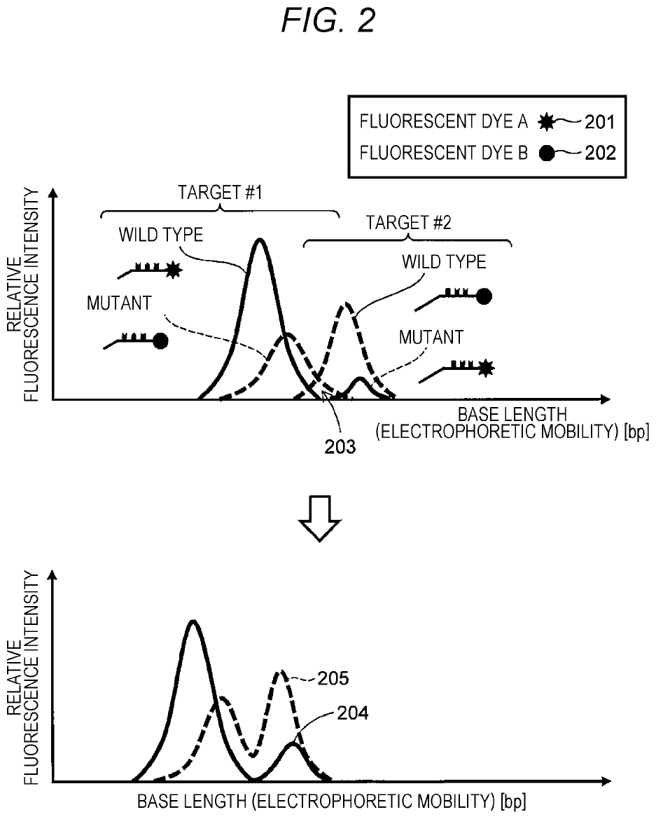

FIG. 2 is an explanatory view showing a state in which fluorescence signals of fluorescent dyes labeled to wild types and mutants of target #1 and target #2 overlap each other because base lengths of primers for detection of target #1 and target #2 are close. Assume that the wild type of target #1 is labeled with fluorescent dye A indicated by 201 and the mutant of target #1 is labeled with fluorescent dye B indicated by 202. In addition, assume that the wild type of target #2 is labeled with fluorescent dye B indicated by 202 and the mutant of target #2 is labeled with fluorescent dye A indicated by 201.

At this time, in order to detect as many target genes as possible in one run (in one reaction), in the case where base lengths of a primer for target #1 and a primer for target #2 are brought close to each other (difference in base length is small), as shown in the upper part of FIG. 2, when the base length is less than a certain base length, overlapping 203 of fluorescence signals occurs. Here, a state in which the wild type of target #1 and the mutant of target #2, which are labeled with fluorescent dye A indicated by 201, overlap each other, and the mutant of target #1 and the wild type of target #2, which are labeled with fluorescent dye B indicated by 202, overlap each other is shown. As abundance of a gene, peak intensity of a fluorescence signal, an integrated value of a fluorescence signal, or the like may be used. Therefore, when there is overlapping 203 of fluorescence signals, quantitativity of abundance of the gene is lost, and a correct genetic mutation amount cannot be calculated.

The lower part of FIG. 2 shows fluorescence signals that occur in the phenomenon in the upper part and are actually observed. Fluorescence signals emitted from fluorescent dyes attached to target #1 and target #2 are detected in an overlapping manner, and regarding a summation signal 204 of fluorescence signals from fluorescent dye A indicated by 201, peak fluorescence signal intensity of the mutant of target #2 is detected higher than that expected under an influence of a fluorescence signal of the wild type of target #1. In addition, when looking at a summation signal 205 of fluorescence signals from fluorescent dye B indicated by 202, peak fluorescence signal intensity of the mutant of target #1 is detected higher than that expected under an influence of a fluorescence signal of the wild type of target #2. In this state, the target mutant gene (for example, a mutant gene derived from a tumor) cannot be accurately quantified.

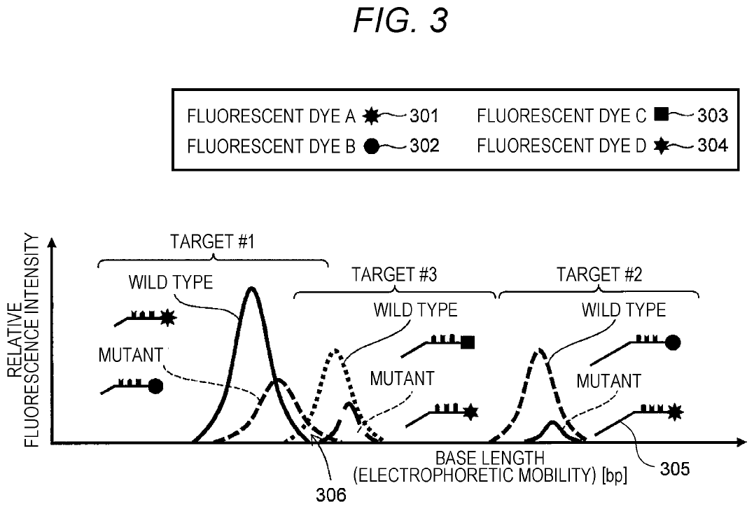

FIG. 3 shows an embodiment of the present invention. Specifically, FIG. 3 is an explanatory view showing a state in which there is no overlapping of fluorescence signals because base lengths (mobility of electrophoresis) of primers for detection of target #1 and target #2 are separated from each other, and even when base lengths of primers for detection of target #1 and target #3 are close to each other, types of fluorescent dyes labeled to wild types and mutants of target #1 and target #3 are different from each other. Assume that the wild type of target #1 is labeled with fluorescent dye A indicated by 301 and the mutant of target #1 is labeled with fluorescent dye B indicated by 302. In addition, assume that the wild type of target #2 is labeled with fluorescent dye B indicated by 302 and the mutant of target #2 is labeled with fluorescent dye A indicated by 301. Furthermore, the wild type of target #3 is labeled with fluorescent dye C indicated by 303 and the mutant of target #3 is labeled with fluorescent dye D indicated by 304. The difference from FIG. 2 is that base lengths (mobility of electrophoresis) of primers for target #1 and target #2 are separated from each other. The base length of the primer 305 for target #2 is long, and the overlapping of the fluorescence signals of fluorescent dye A and fluorescent dye B, which has occurred in FIG. 2, is eliminated.

Regarding label #3, base lengths of primers for target #1 and target #3 are brought close to each other for the purpose of detecting as many target genes as possible in one run. However, since fluorescent dyes of the wild type and the mutant of target #1 are fluorescent dye A (301) and fluorescent dye B (302), respectively, and fluorescent dyes of the wild type and the mutant of target #3 are fluorescent dye C (303) and fluorescent dye D (304), respectively, overlapping 306 of the fluorescence signals can be detected as independent fluorescence signals that do not affect a summation signal of both the fluorescence signals. At this time, a gene target having one wild type and three mutants is also considered. In this case, if primers are designed and arranged so that a fluorescent dye of a wild type of an adjacent target gene and fluorescent dyes of mutants of a gene target having three mutants are different, there is no overlapping of fluorescence signals, and the signals can be distinguished, and distinction between the wild type and the mutants and/or distinction between a certain gene and another gene can be made.

Accordingly, in one aspect, the present invention provides a gene analysis method including: performing a single-base extension reaction using a primer for a single-base extension reaction for detection of a target nucleotide sequence, and a substrate for a single-base extension reaction having a fluorescent dye; subjecting a reaction product of the single-base extension reaction to electrophoresis; and measuring mobility of the electrophoresis and fluorescence intensity of the fluorescent dye, and detecting a wild type and a mutant of the target nucleotide sequence based on the fluorescence intensity, wherein the primer for a single-base extension reaction includes two or more primers having different sequences and different lengths in order to detect two or more target nucleotide sequences, the substrate for a single-base extension reaction having a fluorescent dye includes two or more substrates having different fluorescent dyes, and for the target nucleotide sequences from which the reaction products of the single-base extension reaction that are adjacent as mobility of electrophoresis when measuring the fluorescence intensity are derived, a combination of the primer for a single-base extension reaction and the substrate for a single-base extension reaction is designed so that a fluorescent dye of a substrate incorporated by a single-base extension reaction of a wild type of a first target nucleotide sequence and a fluorescent dye of a substrate incorporated by a single-base extension reaction of a mutant of a second target nucleotide sequence are different.

The present invention is based on a gene analysis method by a combination of a single-base extension reaction and electrophoresis, and such a gene analysis method is well known in the art as described in, for example, Dias-Santagata, D. et al., EMBO Molecular Medicine, Vol. 2, pp. 146-158 (2010); Sanchez, J. J. et al., Electrophoresis, Vol. 27, pp. 1713-1724 (2006); and WO 2016/172571 A.

The single-base extension reaction may be performed using a primer for a single-base extension reaction for detection of a target nucleotide sequence in the presence of a substrate (dideoxynucleotide triphosphate: ddNTP) to which a fluorescent dye is bound.

In the present invention, the target nucleotide sequence refers to a nucleotide sequence for which detection (and quantification) is desired. For example, the target nucleotide sequence may be a nucleotide sequence including a genetic mutation, and the target nucleotide sequence may be detected (and quantified) by distinguishing between a wild type and a mutant. Such genetic mutation may include, but is not limited to, a single nucleotide polymorphism (SNP), an insertion mutation, a deletion mutation, or the like. The mutant of the target nucleotide sequence may include one mutant or a plurality of mutants. For example, there is a genetic mutation in which a wild type and two to three mutants exist, and such a plurality of mutants can also be distinctively detected (and quantified) according to the present invention.

In the present specification, although the target nucleotide sequences are distinguished using the modifiers “first” and “second” for convenience, the target nucleotide sequences are not limited to two types, and n types of target nucleotide sequences are intended to be distinguished from each other.

A test sample to be subjected to the present method may not be particularly limited as long as it is a sample to be detected for a target nucleotide sequence, and may include deoxyribonucleic acid (DNA), for example, genomic DNA, cDNA, and ribonucleic acid (RNA), for example, messenger RNA (mRNA), and fragments thereof. In the present invention, it may be preferable to use, for example, cell-free DNA (cfDNA, DNA free in blood) or circulating tumor DNA (ctDNA) as the test sample. Preparation of nucleic acids from a sample can be performed by a method known in the art. Kits are commercially-available from many manufacturers in order to prepare nucleic acids, and it is possible to simply purify a target nucleic acid.

In addition, a primer for a single-base extension reaction is prepared. The primer for a single-base extension reaction may be either DNA or RNA, and may be selected according to the type of the test sample and the target nucleotide sequence, and the type of polymerase used in the single-base extension reaction. Preferably, the primer may be DNA, and a single-base extension reaction using DNA or mRNA as a template may be performed as the test sample.

In the present invention, the primer for a single-base extension reaction may include two or more primers having different sequences and lengths in order to detect two or more target nucleotide sequences. Since the primer is required for each target nucleotide sequence, primers may be designed in the number corresponding to the type of the target nucleotide sequence to be detected. The primer may include, for example, primers having different sequences and lengths for detection of 20 or more (20 to 100 as an example), preferably 50 or more (50 to 100 as an example) target nucleotide sequences.

Each primer may be designed so as to have a sequence that specifically binds to a target nucleotide sequence, that is, to have a sequence complementary to a target nucleotide sequence. The target nucleotide sequence to be detected may be determined from the sequence of the primer. In addition, the base length of the primer affects the base length (size) of a reaction product of a single-base extension reaction, and thus mobility of electrophoresis may be determined from the length of the primer. In the present invention, it is necessary to appropriately design the sequence and the length of the primer(s).

Primer design methods are well known in the art, and a primer that can be used in the present invention may be designed so as to satisfy conditions allowing specific annealing, for example, so as to have a length and base composition (melting temperature) allowing specific annealing. For example, the length having a function as a primer may be preferably 10 bases or more, more preferably 15 to 50 bases, and still more preferably 15 to 30 bases, for example, about 20 bases. In designing a primer, it may be preferable to confirm a GC content of the primer and melting temperature (Tm) of the primer. Known primer design software can be used to confirm Tm. The designed primer can be chemically synthesized by a known oligonucleotide synthesis method, but is usually synthesized using a commercially available chemical synthesis apparatus.

The primer may have a tag (molecule weight) for adjusting mobility of electrophoresis, for example, an interstrand-crosslinked double-stranded DNA tag. The present inventors have previously developed a fragment analysis method using capillary electrophoresis, and developed an analysis method capable of expanding the number of genetic mutations that can be simultaneously detected to several tens to several hundreds of types. Specifically, by using a primer to which an interstrand-crosslinked double-stranded DNA tag is linked, the length of the primer can be extended by changing the length of the double-stranded DNA tag, and an electrophoretic distance can be stably extended to 120 bp or more, thereby making it possible to increase the number of genetic mutations that can be simultaneously detected. The double-stranded DNA tag has a length that can be distinguished by mobility and has at least one interstrand crosslink. In the present invention, “interstrand crosslinking” means that one strand and the other strand in a double-stranded DNA are crosslinked at least at one position. Such a method of intramolecular crosslinking between two chains is not particularly limited as long as it is a method known in the art. Preferably, the interstrand crosslinking is by photocrosslinking. The double-stranded DNA tag having an interstrand crosslink defines a migration distance (mobility) in electrophoresis. That is, by linking double-stranded DNA tags having different lengths to primers, the migration distance can be changed in electrophoresis. Since a nucleic acid having a length of up to about 600 bases can be detected by capillary electrophoresis, the length of the double-stranded DNA tag can be in a range of 1 to about 590 bases, except for a length (10 to 30 bases) of a primer of a portion that binds to a target nucleotide sequence. The sequence of the double-stranded DNA tag is not particularly limited as long as it is a nucleic acid having an interstrand crosslink. In addition, the double-stranded DNA tag can be chemically synthesized by a known oligonucleotide synthesis method, but is usually synthesized using a commercially available chemical synthesis apparatus.

In the method of the present invention, a single-base extension reaction may be performed using the above-described primer in the presence of a substrate for a single-base extension reaction having a fluorescent dye. The single-base extension reaction is known in the art and is typically a single-base extension reaction using a polymerase. The polymerase to be used may be selected depending on the type of template (test sample) and the type of primer to be used. For example, a DNA-dependent or RNA-dependent DNA polymerase may be used for a single-base extension reaction using a DNA primer using DNA or RNA as a template, respectively.

The single-base extension reaction is widely known in the art, and for example, Dias-Santagata, D. et al., EMBO Molecular Medicine, Vol. 2, pp. 146-158 (2010) and the like describe a method of efficiently elongating one base by a cycle reaction.

When a target nucleotide sequence is present, a primer may hybridize to the target nucleotide sequence, and a nucleotide may be incorporated as a substrate from the 3′ end portion of the primer by a polymerase synthesis reaction. At this time, by using, for example, a dideoxynucleotide (ddNTP) as a nucleotide (substrate) to be incorporated, the synthesis reaction may be terminated only by single base extension.

In the present invention, a substrate having a fluorescent dye may be used as a substrate for a single-base extension reaction, wherein the substrate may include two or more substrates having different fluorescent dyes. The fluorescent dye may be useful for simply detecting whether a substrate has been incorporated or not or for determining the type of base incorporated. A fluorescent dye known in the art can be used. Examples of the fluorescent dye may include, but are not limited to, fluorescein, fluorescein isothiocyanate (FITC), sulforhodamine (TR), tetramethylrhodamine (TRITC), carboxy-X-rhodamine (ROX), carboxytetramethylrhodamine (TAMRA), NED, 5-carboxyfluorescein (5-FAM), 6-carboxyfluorescein (6-FAM), 5′-hexachlorofluorescein CE-phosphoramidite (HEX), 6-carboxy-4′,5′-dichloro-2′,7′-dimethoxyfluorescein (JOE), 5′-tetrachlorofluorescein CE-phosphoramidite (TET), rhodamine 110 (R110), rhodamine 6G (R6G), VIC (registered trademark), ATTO-based, Alexa Fluor (registered trademark)-based, Texas red, and Cy-based, and examples of the fluorescent dye that does not cause a shift in electrophoresis size may include carboxy-dichloro rhodamine 110 (dR110), dihydro rhodamine 6G (dR6G), tetramethyl rhodamine (dTAMRA), and carboxy-X-rhodamine (dROX). For example, when trying to determine the type of base, five types of fluorescent dyes that are excited and detected at different wavelengths can be used in combination to identify five types including four types of bases and a reference (to detect and correct the base length from reference ladder DNA). The type, the introduction method, and the like of such fluorescent dyes may not be particularly limited, and various conventionally known means can be used.

In the present invention, for target nucleotide sequences from which reaction products of a single-base extension reaction that are adjacent as mobility of electrophoresis when measuring fluorescence intensity are derived, a combination of a primer for a single-base extension reaction and a substrate for a single-base extension reaction is designed so that a fluorescent dye of a substrate incorporated by a single-base extension reaction of a wild type of a first target nucleotide sequence and a fluorescent dye of a substrate incorporated by a single-base extension reaction of a mutant of a second target nucleotide sequence may be different. Specifically, as in the detection of target #1 and target #3 in FIG. 3, a combination of each primer and substrate may be designed so that fluorescent dye A 301 resulting from the wild type of target #1 and fluorescent dye D 304 resulting from the mutant of target #3 are different. Alternatively, in FIG. 3, a combination of each primer and substrate may be designed so that fluorescent dye B 302 resulting from the mutant of target #1 and fluorescent dye C 303 resulting from the wild type of target #3 are different. As a result, overlapping 306 of the fluorescence signals can be detected as independent fluorescence signals that do not affect a summation signal of both the fluorescence signals (FIG. 3).

In one embodiment, for target nucleotide sequences from which reaction products of a single-base extension reaction that are adjacent as mobility of electrophoresis when measuring fluorescence intensity are derived, a combination of a primer for a single-base extension reaction and a substrate for a single-base extension reaction may be designed so that a fluorescent dye of a substrate incorporated by a single-base extension reaction of a wild type and a mutant of a first target nucleotide sequence and a fluorescent dye of a substrate incorporated by a single-base extension reaction of a wild type and a mutant of a second target nucleotide sequence are different. Regarding this, for example, in FIG. 3, a combination of each primer and substrate incorporated may be designed so that fluorescent dye A 301 and fluorescent dye B 302 resulting from the wild type and the mutant of target #1 and fluorescent dye C 303 and fluorescent dye D 304 resulting from the wild type and the mutant of target #3 are different.

For example, capillary electrophoresis (CE) apparatuses can detect nucleic acids in a range of about 500 to 600 bp in base length, but the range of electrophoresis that can actually be used quantitatively is about 200 to 300 bases in length. In the conventional art, it has been known to design a primer by providing a difference of at least four bases in length as mobility of electrophoresis, and it has been difficult to detect 50 items as types of target nucleotide sequences that can be simultaneously detected. In the present invention, even when the difference in mobility of electrophoresis is less than 4 bases in length, it is possible to quantitatively detect a plurality of, for example, 20 or more (20 to 100 as an example), preferably 50 or more (50 to 100 as an example) target nucleotide sequences simultaneously without being affected by overlapping of fluorescence signals.

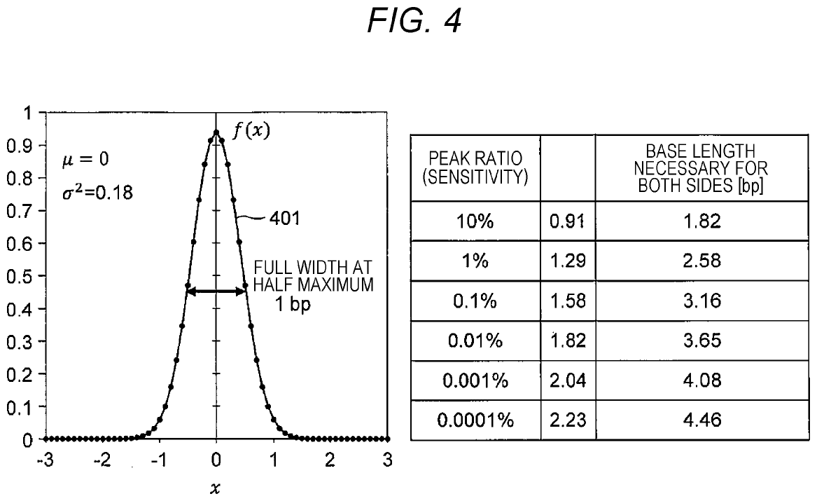

In another embodiment, for target nucleotide sequences from which reaction products of a single-base extension reaction that are adjacent as mobility of electrophoresis when measuring fluorescence intensity are derived, a primer for a single-base extension reaction may be designed so as to adjust a difference in mobility of electrophoresis according to a content ratio of a mutant to a wild type of the target nucleotide sequence. The difference in mobility of electrophoresis can be adjusted, for example, by adjusting the length of the primer and/or by adjusting a tag (molecule weight) added to the primer. This embodiment will be described with reference to FIG. 4.

FIG. 4 is a diagram explaining a correspondence between distance in terms of electrophoretic mobility (base length) and percentage as a fluorescence intensity peak ratio when a fluorescence signal of a labeling fluorescent dye detected by capillary electrophoresis is assumed to be a Gaussian function. The fluorescence signal of the labeling fluorescent dye detected by capillary electrophoresis is assumed to be a Gaussian function f(x) of the following formula (1):

$$f ( x ) = \frac{1}{\sqrt{2 π} σ} exp \{ - \frac{{( x - μ )}^{2}}{2 {σ}^{2}} \} \qquad ( Formula 1 )$$

wherein σ² represents a variance, and μ represents a mean. A signal shape 401 when full width at half maximum FWHM of the following formula (2):

$$FWHM = 2 \sqrt{2 ln 2} \cdot σ \qquad ( Formula 2 )$$

is set to 1 bp is shown in FIG. 4. When considered in terms of the peak ratio of the fluorescence intensity, signal values of 10%, 1%, 0.1%, 0.01%, 0.001%, and 0.0001% as compared with the central peak are obtained at base positions where electrophoretic mobility (base length) is separated by 0.91 bp, 1.29 bp, 1.58 bp, 1.82 bp, 2.04 bp, and 2.23 bp from the center, respectively. Actually, the Gaussian function itself is not formed by tailing of the fluorescence signal, and it is necessary to pay attention to the fact that the half width is also widened.

In order to quantitatively measure an abundance ratio of a mutant to a wild type, it can be determined from the relationship table shown in FIG. 4 how far in base length is required as mobility of primers for adjacent target nucleotide sequences. For example, when quantitative measurement is performed with a detection sensitivity capable of detecting an abundance ratio of a mutant to a wild type of 1%, it is necessary to prevent a fluorescence signal of the adjacent target from overlapping a fluorescence signal peak of a mutation of 1%. Considering a peak ratio (sensitivity) corresponding to 1/100, that is, 0.01% of the fluorescence signal of a mutation of 1% as a level at which the signal is not affected, it is necessary to separate the base length by 1.82 bp or more. Considering that the Gaussian function spreads on both sides of the graph, it can be determined that it is preferable to design adjacent primers so as to be separated by 3.65 bp or more, which is twice 1.82 bp, as the length of the primers for adjacent target nucleotide sequences. Note that a shape of the fluorescence signal may also change depending on electrophoresis conditions (injection voltage, injection time, electrophoresis voltage, electrophoresis time, temperature, and the like) and specifications of a capillary electrophoresis apparatus itself (theoretically, both are Gaussian functions, but the variance, the full width at half maximum, and the like may be different).

As described above, when an abundance ratio of a mutant to a wild type can be predicted in advance, a primer(s) can be designed based on the abundance ratio so as not to affect a fluorescence signal. For example, when it can be predicted in advance that a content ratio of a mutant to a wild type is 0.01% or more, there may be no influence on a fluorescence signal if a primer is designed so that 4.46 bases corresponding to 1/100 (0.0001%) of 0.01% are separated. Therefore, in one embodiment, for a first target nucleotide sequence in which a content ratio of a mutant to a wild type is in a range of 0.01% to 10%, primers for a single-base extension reaction may be designed so that a difference as mobility of electrophoresis between a primer for a single-base extension reaction for detection of a second target nucleotide sequence and a primer for a single-base extension reaction for detection of a first target nucleotide sequence may be at least 4.46 to 3.16 bases. In addition, for example, for a first target nucleotide sequence in which a content ratio of a mutant to a wild type is in a range of 0.1% to 10%, primers for a single-base extension reaction may be designed so that a difference as mobility of electrophoresis between a primer for a single-base extension reaction for detection of a second target nucleotide sequence and a primer for a single-base extension reaction for detection of a first target nucleotide sequence may be at least 4.08 to 3.16 bases. In the conventional method of quantitatively measuring a ratio of mutants to wild types of a plurality of target genes, a mutant allele frequency is typically detected up to about 5%, but quantitative measurement of less than 10% can be effectively performed by using the gene analysis method of the present invention.

After the single-base extension reaction, the obtained reaction product(s) is/are subjected to electrophoresis and analyzed. The electrophoresis may not be particularly limited as long as it is a measurement method capable of fragment analysis by electrophoresis, and for example, capillary electrophoresis (CE) or electrophoresis in a microchannel such as Micro-Electro-Mechanical Systems (MEMS) can be used. In a preferred embodiment, the electrophoresis may be capillary electrophoresis (CE).

Electrophoresis, for example, CE is a method of separating loaded components by a difference in mobility based on charge, size, shape, and the like. Based on mobility, the type of target nucleotide sequence (based on the type of primer) can be identified. In addition, based on a signal of a fluorescent dye, the presence or absence of a target nucleotide sequence or the type of specific base in a target nucleotide sequence (based on the type of substrate incorporated by a single-base extension reaction), for example, a wild type and a mutant can be discriminated.

In one embodiment, detecting a wild type and a mutant of a target nucleotide sequence of the method according to the present invention may include quantifying a content ratio between the wild type and the mutant of the target nucleotide sequence based on magnitude of fluorescence intensity. In this case, for example, an abundance ratio of a mutant sequence to a wild-type sequence required for cancer diagnosis or a frequency of genetic mutation can be quantified. In one embodiment, when a plurality of target nucleotide sequences to be analyzed may include a wild-type sequence and a mutant sequence, and a content ratio of the mutant sequence to the wild-type sequence is in a range of 0.01% to 10%, for example, in a range of 0.01% to 1%, and further in a range of 0.01% to 0.1%, the target nucleotide sequences can be analyzed. In this way, target nucleotide sequences can be subjected to quantitative gene analysis.

When a target nucleotide sequence for which quantitative gene analysis is desired to be performed is included, a primer for a single-base extension reaction for detection of a target nucleotide sequence for which a content ratio between a wild type and a mutant is quantified may be designed so as to have a shorter length than that of other primers for a single-base extension reaction. Due to a nature of fragment analysis, a resolution of electrophoresis tends to decrease as the number of bases increases, and therefore detection accuracy can be improved by using a short-base primer for a target gene for which more accurate quantitativity is required.

In another aspect, the present invention provides a gene analysis method including: performing a single-base extension reaction using a primer for a single-base extension reaction for detection of a target nucleotide sequence, and a substrate for a single-base extension reaction having a fluorescent dye; subjecting a reaction product of the single-base extension reaction to electrophoresis; and measuring mobility of the electrophoresis and fluorescence intensity of the fluorescent dye, and detecting a wild type and a mutant of the target nucleotide sequence based on the fluorescence intensity, wherein the primer for a single-base extension reaction includes two or more primers having different sequences and different lengths in order to detect two or more target nucleotide sequences, the substrate for a single-base extension reaction having a fluorescent dye includes two or more substrates having different fluorescent dyes, and for the target nucleotide sequences from which the reaction products of the single-base extension reaction that are adjacent as mobility of electrophoresis when measuring the fluorescence intensity are derived, the primer for a single-base extension reaction is designed so as to adjust a difference in the mobility of the electrophoresis according to a content ratio of the mutant to the wild type of the target nucleotide sequence.

This aspect of the present invention can be implemented similarly to the embodiments of the above aspect. In one aspect, for target nucleotide sequences from which reaction products of a single-base extension reaction that are adjacent as mobility of electrophoresis when measuring fluorescence intensity are derived, a combination of a primer for a single-base extension reaction and a substrate for a single-base extension reaction can be designed so that a fluorescent dye of a substrate incorporated by a single-base extension reaction of a wild type of a first target nucleotide sequence and a fluorescent dye of a substrate incorporated by a single-base extension reaction of a mutant of a second target nucleotide sequence may be different.

The above-described gene analysis method according to the present invention can be simply and quickly performed by a kit for gene analysis containing necessary components.

Therefore, in another aspect, the present invention provides a kit for gene analysis for use in the gene analysis method according to the present invention, including: a primer for a single-base extension reaction for detection of a target nucleotide sequence; and a substrate for a single-base extension reaction having a fluorescent dye, wherein the primer for a single-base extension reaction includes two or more primers having different sequences and different lengths in order to detect two or more target nucleotide sequences, the substrate for a single-base extension reaction having a fluorescent dye includes two or more substrates having different fluorescent dyes, and for the target nucleotide sequences from which reaction products of a single-base extension reaction that are adjacent as mobility of electrophoresis are derived, a combination of the primer for a single-base extension reaction and the substrate for a single-base extension reaction is designed so that a fluorescent dye of a substrate incorporated by a single-base extension reaction of a wild type of the first target nucleotide sequence and a fluorescent dye of a substrate incorporated by a single-base extension reaction of a mutant of the second target nucleotide sequence are different.

In yet another aspect, the present invention provides a kit for gene analysis for use in the gene analysis method according to the present invention, including: a primer for a single-base extension reaction for detection of a target nucleotide sequence; and a substrate for a single-base extension reaction having a fluorescent dye, wherein the primer for a single-base extension reaction includes two or more primers having different sequences and different lengths in order to detect two or more target nucleotide sequences, the substrate for a single-base extension reaction having a fluorescent dye includes two or more substrates having different fluorescent dyes, and for the target nucleotide sequences from which reaction products of a single-base extension reaction that are adjacent as mobility of electrophoresis are derived, the primer for a single-base extension reaction is designed so as to adjust a difference in the mobility of the electrophoresis according to the content ratio of the mutant to the wild type of the target nucleotide sequence.

The kit according to the present invention may contain, in addition to the above components, a buffer constituting a reaction liquid, enzymes (polymerase, reverse transcriptase, and the like), a standard sample for calibration, and the like. By providing a primer and a substrate used in the single-base extension reaction as a kit, it may be possible to more quickly and simply perform gene analysis.

Hereinafter, the present invention will be specifically described with reference to examples, but these examples are merely provided for the purpose of describing the present invention, and do not limit or restrict the scope of the invention disclosed in this application.

### EXAMPLES

As a standard sample containing cancer-related genetic mutations, OncoSpan DNA Reference Standard (Horizon) was used, and EGFR L858, which is a type of cancer driver gene, was used as a target gene. The mutation of EGFR L858 is a sequence EGFR L858R in which the 858th leucine (L: CUG) is substituted with arginine (R: CGG) by one base. First, PCR for cloning was performed using the above standard sample containing EGFR L858 wild-type (EGFR L858WT) and mutant (L858R) genes as a template. The PCR product was transformed into E. coli, cultured in an LB medium, and then amplified by colony direct PCR. A sequencing reaction was performed using BigDye Terminator Sequencing Kit (Thermo Fisher Scientific, Inc.), and after purification, the sequence was confirmed by a genetic analyzer SeqStudio, and then a plasmid was extracted.

Using the extracted plasmid as a template (a target tumor-derived gene sequence), 0.2 μM of the EGFR L858 primer shown in the following table, 1 U of DNA polymerase, and ddNTPs modified with fluorescent dyes (T label: ROX-ddUTP, G label: R110-ddGTP) (PerkinElmer, Inc.) were each mixed, and a single-base extension reaction was performed under the condition of [96° C.×10 seconds→50° C.×5 seconds→60° C.×30 seconds]×25 cycles by a thermal cycler. The concentration of the target template DNA was wild type: mutant=100 fmol: 1 fmol (mutant content ratio: 1%).

TABLE 1

| | Detectable base | | |
|---|---|---|---|
| Sequence (5′→3′) | Primer | | |
| Non-italic: SP6 promoter (24 mer) | size | Wild | |
| Italic: Target gene sequence | (bp) | type | Mutant |
| ATGGGTGGACTTTAGGTGACACTATAGCAGCATGTCAAGATCAC | 55 | T | G |
| AGATTTTGGGC (SEQ ID NO: 1) | | | |

In the sequence:
Bold highlight: Universal sequence for base length adjustment
Non-italic: SP6 promoter (24 mer)
Italic: Target gene sequence

After the single-base extension reaction, a dephosphorylation reaction (SAP) treatment was performed in order to prevent interference by a fluorescently labeled ddNTP which is an unreacted substrate. 1 μL of SAP was added to 10 μL of the reaction product, and the mixture was reacted at 37° C. for 1 hour and then at 75° C. for 15 minutes. The sample after the SAP treatment, a size marker, and Hi-Di Formamide were mixed, and after heat treatment at 95° C.×5 minutes, fragment analysis was performed using a CE sequencer DS3000 (Hitachi High-Tech Corporation).

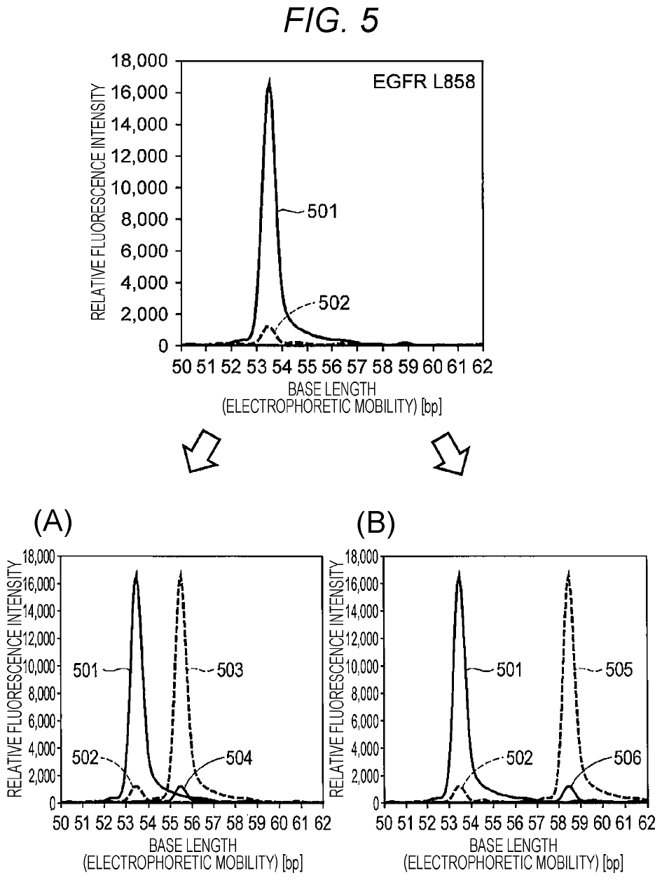

FIG. 5 is a diagram showing results of fluorescence signal intensity of a wild type and a mutant when the genetic mutation EGFR L858R is targeted. Regarding a fluorescence signal 501 of the wild type of EGFR L858 and a fluorescence signal 502 of the mutant of EGFR L858, fluorescence signal intensity corresponding to wild type: mutant=100 fmol: 1 fmol is detected. Based on this result, the following is assumed.

The lower part (A) of FIG. 5 is a diagram showing superposition of a shape of the same fluorescence signal when it is assumed that a product derived from another gene target primer is adjacent and separated by 2 bp in base length when migrated. Looking at a fluorescence signal 503 of the wild type when the base length of another adjacent gene target primer is separated by 2 bp and a fluorescence signal 504 of the mutant when the base length of another adjacent gene target primer is separated by 2 bp, fluorescence intensity peaks overlap when the fluorescence signal of the latter mutant has the same fluorescent dye as the fluorescence signal 501 of the wild type of EGFR L858, so that the value of the fluorescence intensity peak may not be correctly detected. In this case, by making the fluorescence signals 502 and 503 and the fluorescence signals 501 and 504 in which the fluorescence signals overlap each other be derived from different fluorescent dyes, that is, by labeling bases (ddNTPs) incorporated into the respective primers with different fluorescent dyes (for example, labeling in the same manner as for target #1 and target #3 in FIG. 3), the respective fluorescence signals can be quantitatively detected.

Alternatively, as shown in the lower part (B) of FIG. 5, this is a diagram showing superposition of a shape of the same fluorescence signal when respective primers are designed so that a product derived from another gene target primer is adjacent and separated by 5 bp in base length when migrated. A fluorescence signal 505 of the wild type when the base length of another adjacent gene target primer is separated by 5 bp and a fluorescence signal 506 of the mutant when the base length of another adjacent gene target primer is separated by 5 bp do not overlap with the fluorescence signal 501 of the wild type of EGFR L858 and the fluorescence signal 502 of the mutant of EGFR L858, so that the value of the fluorescence intensity peak may be correctly detected.

As described above, by making fluorescent dyes of gene target primers that are adjacent when migrated different and/or appropriately setting (designing) the length of gene target primers, quantitative measurement of gene targets having no mutual influence can be performed. By setting (designing) a primer length according to a ratio of a mutant to be quantified, it may be possible to detect more items (target nucleotide sequences) with adjacent primers while bringing the items as close as possible.

Incidentally, x-rhodamine (ROX) and rhodamine 110 (R110) are used as the fluorescent dyes used in the above Examples, but the fluorescent dye referred to in the present invention may not be limited thereto, and a fluorescent dye that is labeled to a nucleic acid probe may be generally used. In addition to other derivatives of rhodamine such as rhodamine 6G (R6G) and tetramethylrhodamine (TAMRA), examples of the fluorescent dye may include fluorescein or fluorescein isothiocyanate (FITC), which is a derivative thereof, Alexa 488, Alexa 532, cy3, cy5, and Texas red. The fluorescent dye can be appropriately determined according to an excitation wavelength of a laser beam mounted on a capillary electrophoresis apparatus to be used.

The present invention is not limited to the above-described embodiments, and includes various modifications. For example, the above-described examples have been described in detail in order to describe the present invention in an easy-to-understand manner, and are not necessarily limited to those having all the described configurations. In addition, a part of the configuration of a certain example can be replaced with the configuration of another example, and the configuration of another example can be added to the configuration of a certain example. In addition, it is possible to add, delete, and replace other configurations for a part of the configuration of each example.

### SEQUENCE LISTING FREE TEXT

SEQ ID NO: 1: DNA (synthetic oligonucleotide)

What is claimed is:

1. A gene analysis method comprising:
performing a single-base extension reaction using a primer for a single-base extension reaction for detection of a target nucleotide sequence, and a substrate for a single-base extension reaction having a fluorescent dye;
subjecting a reaction product of the single-base extension reaction to electrophoresis; and
measuring mobility of the electrophoresis and fluorescence intensity of the fluorescent dye, and detecting a wild type and a mutant of the target nucleotide sequence based on the fluorescence intensity,
wherein the primer for a single-base extension reaction comprises two or more primers having different sequences and different lengths in order to detect two or more target nucleotide sequences,
the substrate for a single-base extension reaction having a fluorescent dye comprises two or more substrates having different fluorescent dyes, and
for the target nucleotide sequences from which the reaction products of the single-base extension reaction that are adjacent as mobility of electrophoresis when measuring the fluorescence intensity are derived, a combination of the primer for a single-base extension reaction and the substrate for a single-base extension reaction is designed so that a fluorescent dye of a substrate incorporated by a single-base extension reaction of a wild type of a first target nucleotide sequence and a fluorescent dye of a substrate incorporated by a single-base extension reaction of a mutant of a second target nucleotide sequence are different.

2. The method according to claim 1, wherein, for the target nucleotide sequences from which the reaction products of the single-base extension reaction that are adjacent as mobility of electrophoresis when measuring the fluorescence intensity are derived, a combination of the primer for a single-base extension reaction and the substrate for a single-base extension reaction is designed so that a fluorescent dye of a substrate incorporated by a single-base extension reaction of a wild type and a mutant of the first target nucleotide sequence and a fluorescent dye of a substrate incorporated by a single-base extension reaction of a wild type and a mutant of the second target nucleotide sequence are different.

3. The method according to claim 1, wherein the detecting a wild type and a mutant of the target nucleotide sequence comprises quantifying a content ratio between the wild type and the mutant of the target nucleotide sequence based on magnitude of the fluorescence intensity.

4. The method according to claim 3, wherein the primer for a single-base extension reaction for detection of the target nucleotide sequence for which the content ratio between the wild type and the mutant is quantified is designed so as to have a shorter length than that of other primers for a single-base extension reaction.

5. The method according to claim 1, wherein analysis is performed for the target nucleotide sequence in which the content ratio of the mutant to the wild type is in a range of 0.01% to 10%.

6. The method according to claim 1, wherein the two or more target nucleotide sequences comprise 20 or more target nucleotide sequences.

7. The method according to claim 1, wherein the mutant of the target nucleotide sequence comprises a plurality of mutants.

8. The method according to claim 1, wherein, for the target nucleotide sequences from which the reaction products of the single-base extension reaction that are adjacent as mobility of electrophoresis when measuring the fluorescence intensity are derived, the primer for a single-base extension reaction is designed so as to adjust a difference in the mobility of the electrophoresis according to the content ratio of the mutant to the wild type of the target nucleotide sequence.

9. The method according to claim 8, wherein, for the first target nucleotide sequence in which the content ratio of the mutant to the wild type is in a range of 0.01% to 10%, the primer for a single-base extension reaction is designed so that a difference as mobility of electrophoresis between a primer for a single-base extension reaction for detection of the second target nucleotide sequence and a primer for a single-base extension reaction for detection of the first target nucleotide sequence is at least 4.46 to 3.16 bases.

10. A gene analysis method comprising:
performing a single-base extension reaction using a primer for a single-base extension reaction for detection of a target nucleotide sequence, and a substrate for a single-base extension reaction having a fluorescent dye;
subjecting a reaction product of the single-base extension reaction to electrophoresis; and
measuring mobility of the electrophoresis and fluorescence intensity of the fluorescent dye, and detecting a wild type and a mutant of the target nucleotide sequence based on the fluorescence intensity,
wherein the primer for a single-base extension reaction comprises two or more primers having different sequences and different lengths in order to detect two or more target nucleotide sequences,
the substrate for a single-base extension reaction having a fluorescent dye comprises two or more substrates having different fluorescent dyes, and
for the target nucleotide sequences from which the reaction products of the single-base extension reaction that are adjacent as mobility of electrophoresis when measuring the fluorescence intensity are derived, the primer for a single-base extension reaction is designed so as to adjust a difference in the mobility of the electrophoresis according to the content ratio of the mutant to the wild type of the target nucleotide sequence.

11. The method according to claim 10, wherein, for the target nucleotide sequences from which the reaction products of the single-base extension reaction that are adjacent as mobility of electrophoresis when measuring the fluorescence intensity are derived, a combination of the primer for a single-base extension reaction and the substrate for a single-base extension reaction is designed so that a fluorescent dye of a substrate incorporated by a single-base extension reaction of a wild type of the first target nucleotide sequence and a fluorescent dye of a substrate incorporated by a single-base extension reaction of a mutant of the second target nucleotide sequence are different.

12. A kit for gene analysis for use in the method according to claim 1, comprising:
a primer for a single-base extension reaction for detection of a target nucleotide sequence; and
a substrate for a single-base extension reaction having a fluorescent dye,
wherein the primer for a single-base extension reaction comprises two or more primers having different sequences and different lengths in order to detect two or more target nucleotide sequences,
the substrate for a single-base extension reaction having a fluorescent dye comprises two or more substrates having different fluorescent dyes, and
for the target nucleotide sequences from which reaction products of a single-base extension reaction that are adjacent as mobility of electrophoresis are derived, a combination of the primer for a single-base extension reaction and the substrate for a single-base extension reaction is designed so that a fluorescent dye of a substrate incorporated by a single-base extension reaction of a wild type of the first target nucleotide sequence and a fluorescent dye of a substrate incorporated by a single-base extension reaction of a mutant of the second target nucleotide sequence are different.


SEQUENCE LISTING (1 sequence)

SEQ ID NO: 1 - DNA, 55 bp, synthetic construct
  misc_feature 1..55: note = synthetic oligonucleotide
  source 1..55: mol_type = other DNA
atgggtggactttaggtgacactatagcagcatgtcaagatcacagattttgggc